(19) United States
(12) Patent Application Publication
(10) Pub. No.: US 2022/0330903 A1
(43) Pub. Date: Oct. 20, 2022

(54) IDENTIFYING NEAR-FALL EVENTS BASED ON INERTIAL MEASUREMENT UNIT DATA

(71) Applicant: Regents of the University of Minnesota, Minneapolis, MN (US)

(72) Inventors: Robert McGovern, Edina, MN (US); Ali Nouriani, Minneapolis, MN (US); Rajesh Rajamani, Minneapolis, MN (US)

(73) Assignee: Regents of the University of Minnesota, Minneapolis, MN (US)

(21) Appl. No.: 17/723,912

(22) Filed: Apr. 19, 2022

Related U.S. Application Data
(60) Provisional application No. 63/176,469, filed on Apr. 19, 2021.

(51) Int. Cl. A61B 5/00; A61B 5/11; A61B 5/16

(52) U.S. Cl. CPC A61B 5/7282; A61B 5/1126; A61B 5/6823; A61B 5/6828; A61B 5/162; A61B 5/7264; A61B 5/1114; A61B 5/1117; A61B 2562/0219; A61B 2562/04

(57) ABSTRACT

A sensing and processing system includes a plurality of wearable sensing devices each including an inertial measurement unit (IMU) to be positioned on a subject and generate accelerometer signals and gyroscope signals. The system includes a processor to identify near-fall events indicative of the subject nearly falling down based on the generated accelerometer signals and gyroscope signals, and wherein the processor is to generate, for each of the near-fall events, subject response data indicative of a recovery response of the subject to recover from the near-fall event.

### RELATED APPLICATIONS

This Non-Provisional patent application claims the benefit of the filing date of U.S. Provisional Patent Application No. 63/176,469, filed Apr. 19, 2021, entitled “IDENTIFYING NEAR-FALL EVENTS BASED ON INERTIAL MEASUREMENT UNIT DATA,” the entire teachings of which are incorporated herein by reference.

### BACKGROUND

Falls are a major cause of worsened quality of life, diminished mobility, disability and death in patients with Parkinson's Disease (PD). Methods of measuring balance in PD patients typically occur in a clinic or laboratory. However, since the vast majority of falls occur at home, tracking patients in the clinic or laboratory is not likely to yield the highest quality information to best predict which patients will fall in the future. Because falls are due to a complex, multifactorial set of variables, fall prediction models are typically quite poor.

Some methods of tracking participants at home may involve using mobile phone-based systems such as accelerometers on iPhones. This yields data that is able to generally track activities such as walking, sitting and sleeping, but does not provide quantitative insights into participants' responses when experiencing a fall or near-fall. More elaborate systems using multiple cameras throughout a person's home in order to track their movements may also be used, but this may not be feasible on a wide scale due to its cost, complexity and privacy concerns.

For these and other reasons, a need exists for the present invention.

### SUMMARY

One example is directed to a sensing and processing system, which includes a plurality of wearable sensing devices each including an inertial measurement unit (IMU) to be positioned on a subject and generate accelerometer signals and gyroscope signals. The system includes a processor to identify near-fall events indicative of the subject stumbling or nearly falling down based on the generated accelerometer signals and gyroscope signals, and wherein the processor is to generate, for each of the near-fall events, subject response data indicative of a recovery response of the subject to recover from the near-fall event.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is a block diagram illustrating a sensing and processing system to sense and analyze fall and near-fall events according to an example.

FIG. 2 is a block diagram illustrating elements of the sensing device shown in FIG. 1 according to an example.

FIGS. 3A-3D are diagrams illustrating various views of the sensing device shown in FIG. 1 according to an example.

FIG. 4 is a schematic diagram illustrating a wearable three-sensor configuration of sensing devices for a human subject according to an example.

FIG. 5 is a schematic diagram illustrating a wearable five-sensor configuration of sensing devices for a human subject according to an example.

FIG. 6 is a diagram illustrating an activity classification decision tree using support vector machines (SVMs) for detection of a fall or near-fall event followed by balancing steps according to an example.

FIG. 7 is a diagram illustrating a graph of results from approximately nine minutes of random activities by a human subject instrumented with three IMUs (one each on the chest and two legs) according to an example.

FIG. 8 is a diagram illustrating a leg of a human subject with a first IMU positioned on a shank of the leg and a second IMU positioned on the thigh of the leg according to an example.

FIG. 9 is a diagram illustrating the axes of the IMU and the attachment of it to a body limb segment according to an example.

FIG. 10 is a diagram illustrating a graph of an example RMS signal of a right foot acceleration and its local maximum that are used to identify the heel-strikes of each step according to an example.

FIG. 11 is a schematic diagram illustrating a model of the body and the angles of each limb during in-plane walking according to an example.

FIG. 12 is a diagram illustrating graphs of IR camera measurements and shank tilt angle estimations of a nonlinear observer according to an example.

FIG. 13 is a diagram illustrating graphs of a comparison of step length (e.g., horizontal displacement) estimation of the three observer-based methods with an IR camera according to an example.

FIG. 14 is a diagram illustrating graphs of the estimated bias from the nonlinear observer for the horizontal axis accelerations according to an example.

FIG. 15 is a diagram illustrating a graph of the horizontal displacement error for the right foot for the three observer-based methods (i.e., two-sensor integrator-based, four-sensor angle-based, and three-sensor polynomial angle-based) by comparing with the IR camera according to an example.

FIG. 16 is a diagram illustrating a graph of two outputs and with assumed constant-gain operating regions of the nonlinear observer according to an example.

FIG. 17 is a diagram illustrating graphs of shank and thigh tilt angles according to an example.

FIG. 18 is a diagram illustrating an LSTM cell according to an example.

FIGS. 19A and 19B are block diagrams illustrating activity recognition methods using a CNN-LSTM according to an example.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

### I. Sensing and Processing System

Examples disclosed herein are directed to a sensing and processing system to sense and analyze fall and near-fall events. Some systems may be focused on either detecting a fall of a human subject after it has occurred (i.e., in order to alert emergency responders), or on detecting an imminent fall just before it occurs. In contrast, some examples disclosed herein are directed to automatically detecting a near-fall and characterizing the response of the subject in recovering from the near fall.

Since the vast majority of falls of Parkinson's Disease (PD) patients occur at home, tracking patients in their home environment is likely to yield the highest quality information to best predict which patients will fall in the future. Because falls are due to a complex, multifactorial set of variables, fall prediction models are typically quite poor. Examples disclosed herein use a high quality model that includes the number and type of near-fall events experienced in a subject's home environment, the event preceding the near-falls, and the patient's postural response after the near-fall. Some examples disclosed herein are directed to obtaining data from PD patients using inexpensive wearable sensors that may be used to automatically identify relevant postural instability events and characterize the specific responses to near falls in the home environment, while also preserving privacy and avoiding the use of video cameras.

Some examples disclosed herein are directed to a sensing and processing system that includes a wearable sensor system to monitor a human subject, such as a patient with PD, at home in order to obtain data that may be used to predict the subject's future risk of falls. In some examples, the wearable sensor system includes three sensing devices, which each include an inertial measurement unit (IMU). The subject can affix the three sensing devices on their body at three locations, such as the chest and each shank (i.e., lower leg between the ankle and knee) of the subject. Some examples may utilize more than three sensing devices, with the additional sensing devices affixed to other locations on the subject's body, such as a sensing device on each thigh of the subject.

Sensor data from the sensing devices may be collected over a period of one week, for example, while the subject is at home engaging in typical daily living activities. In some examples, the collected sensor data may then be uploaded to a processing system, such as a cloud-based processing system. In some examples, cloud-based software may be used to analyze the collected data, automatically identify portions of the data where a near-fall event was experienced, extract variables related to the recovery from the near-fall event including the number of balancing steps, step lengths, response time, and additional variables on chest velocity and acceleration that indicate the severity of the event. These response variables may be used to compute the subject's fall risk over a period of time, such as a one-year period, as well as to develop specific medical interventions that can improve the subject's mobility and quality of life. Analysis of the data may also provide a quantitative measure of the health of the subject with respect to their postural instability. In some examples, the collected sensor data may be stored on a SD card or other memory located on the wearable sensing unit for processing after the data collection period is over.

Examples disclosed herein may include various sensor configurations, and may use methods for automatic identification of near-fall events and for estimating variables related to the recovery response and fall severity of the events. In addition to home-based monitoring, examples disclosed herein may also be used in other environments, such as while conducting in-clinic pull tests. Besides using examples disclosed herein for patients with PD, some examples may be useful for other types of subjects, such as patients with other movement disorders, including hydrocephalus and age-related postural instability. In addition to enabling home-based monitoring of PD patients, examples disclosed herein may also be used to replace more expensive infrared camera capture systems that may be utilized by scientists to study postural instability or video-based systems that may be used by physicians to conduct pull tests in a clinic.

FIG. 1 is a block diagram illustrating a sensing and processing system 100 to sense and analyze fall and near-fall events according to an example. System 100 includes a plurality of sensing devices 102(1)-102(3) (collectively referred to as sensing devices 102) and computing device 104. Computing device 104 includes processor 106, memory 108, and wireless transceiver 112. Memory 108 stores sensor data processing module 108. In an example, sensing devices 102 are wearable devices that may be worn by a user. The sensing devices 102 provide sensing data to computing device 104.

Depending on the exact configuration and type of computing device, the memory 108 may be volatile (such as RAM), non-volatile (such as ROM, flash memory, etc.), or some combination of the two. The memory 108 used by computing device 104 is an example of computer storage media (e.g., non-transitory computer-readable storage media storing computer-executable instructions for performing a method). Computer storage media used by computing device 104 according to one example includes volatile and nonvolatile, removable and non-removable media implemented in any suitable method or technology for storage of information such as computer readable instructions, data structures, program modules or other data. Computer storage media includes, but is not limited to, RAM, ROM, EEPROM, SD cards, flash memory or other memory technology, CD-ROM, digital versatile disks (DVD) or other optical storage, magnetic cassettes, magnetic tape, magnetic disk storage or other magnetic storage devices, or any other medium that can be used to store the desired information and that can be accessed by processor 106.

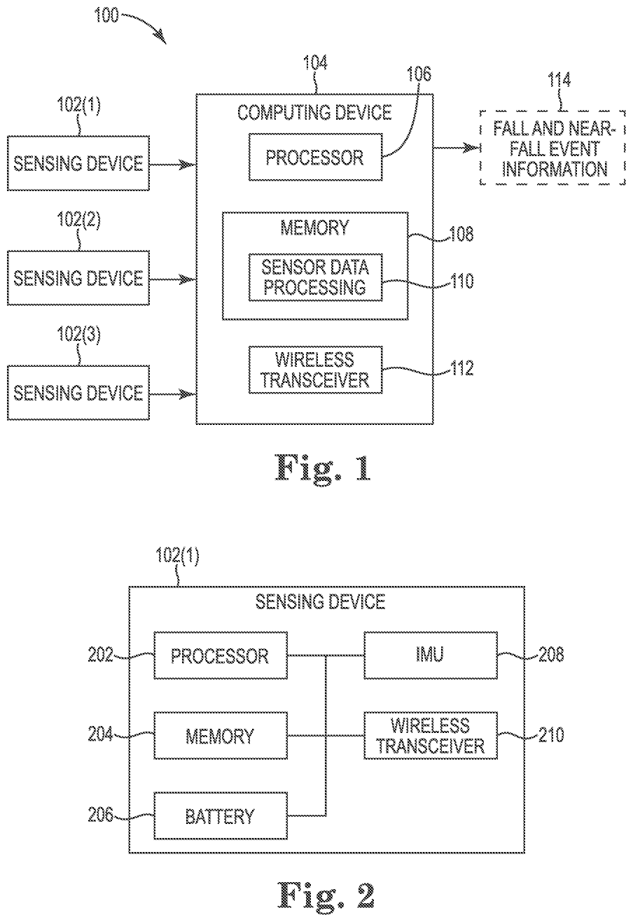

FIG. 2 is a block diagram illustrating elements of the sensing device 102(1) shown in FIG. 1 according to an example. In an example, sensing devices 102(2) and 102(3) (FIG. 1) include the same elements as shown for sensing device 102(1). Sensing device 102(1) includes processor 202, memory 204, battery 206, inertial measurement unit (IMU) 208, and wireless transceiver 210. In an example, memory 204 includes a micro-SD memory card. Battery 206 provides power for processor 202, memory 204, IMU 208, and wireless transceiver 210.

IMU 208 generates IMU sensor data, which may be stored in memory 204 and/or transmitted to computing device 104 via wireless transceiver 210. In an example, the sensor data represents sensed movement of a human subject wearing the sensing devices 102. Computing device 104 receives the transmitted IMU sensor data via wireless transceiver 112. In an example, computing device 104 is a cloud-based device positioned remotely from sensing devices 102, and wireless transceiver 210 is a cellular transceiver that transmits the IMU sensor data to the cloud-based computing device 104 via cellular communications. In another example, computing device 104 is a local device positioned near (e.g., within the same building) as sensing devices 102, and wireless transceiver 210 transmits the IMU sensor data to the local computing device 104 via Bluetooth, Wi-Fi, or other wireless communications protocol.

The IMU 208 of each of the sensing devices 102 may be implemented as an IMU sensor chip that includes a 3-axis accelerometer and a 3-axis gyroscope. Thus, each IMU 208 provides six measurement signals (i.e., three accelerations from the three accelerometers, and three rotational rates from the three gyroscopes).

Processor 106 executes sensor data processing module 110 to perform sensor data processing methods disclosed herein using the sensor information received from sensing devices 102. Sensor data processing module 110 outputs fall and near-fall event information 114, based on the processing of the received sensor information.

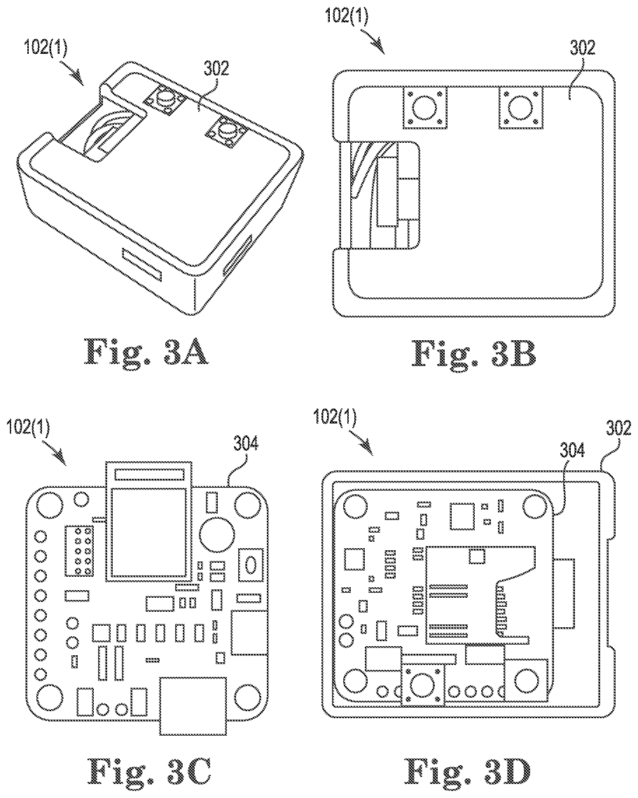

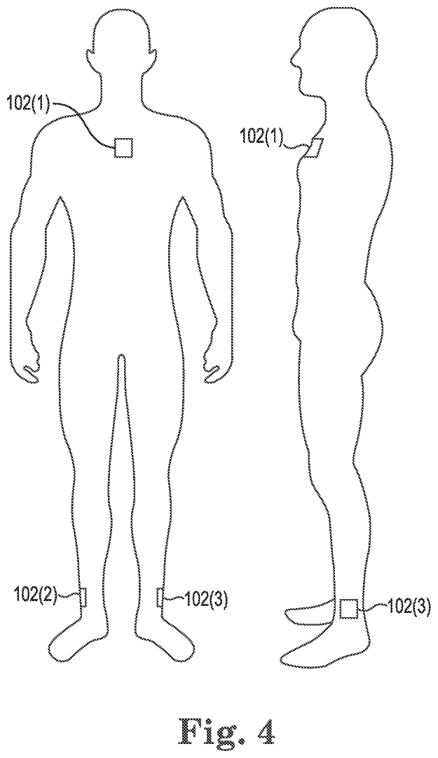

FIGS. 3A-3D are diagrams illustrating various views of the sensing device 102(1) shown in FIG. 1 according to an example. In an example, sensing devices 102(2) and 102(3) (FIG. 1) are configured in the same manner as shown for sensing device 102(1). As shown in FIGS. 3A-3D, the components of the sensing device 102(1) are positioned on a sensor board 304 that is enclosed in a three-dimensional (3D) printed box 302.

FIG. 4 is a schematic diagram illustrating a wearable three-sensor configuration of sensing devices 102(1)-102(3) for a human subject according to an example. As shown in FIG. 4, three sensing devices 102(1)-102(3) are positioned on the subject. One of the sensing devices 102(2) is positioned just above the ankle on the subject's right leg. A second one of the sensing devices 102(3) is positioned just above the ankle on the subject's left leg. A third one of the sensing devices 102(1) is positioned on the chest of the subject. In an example, the sensing devices 102 do not need to be precisely located or precisely aligned. Examples disclosed herein may automatically calibrate and compensate the sensor signals to account for the unknown alignment of the sensing devices 102.

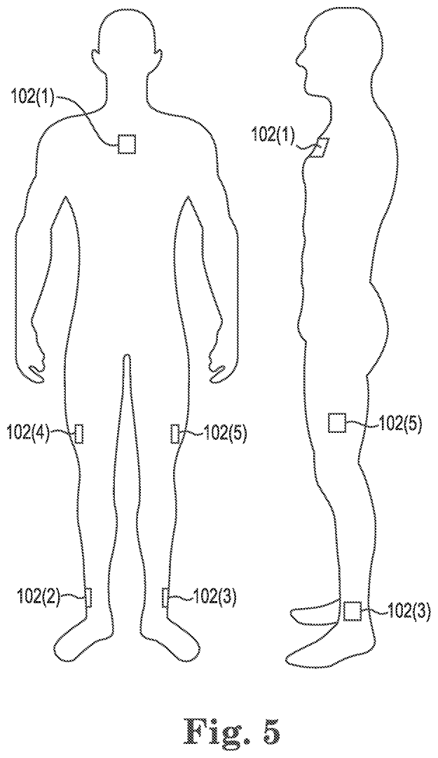

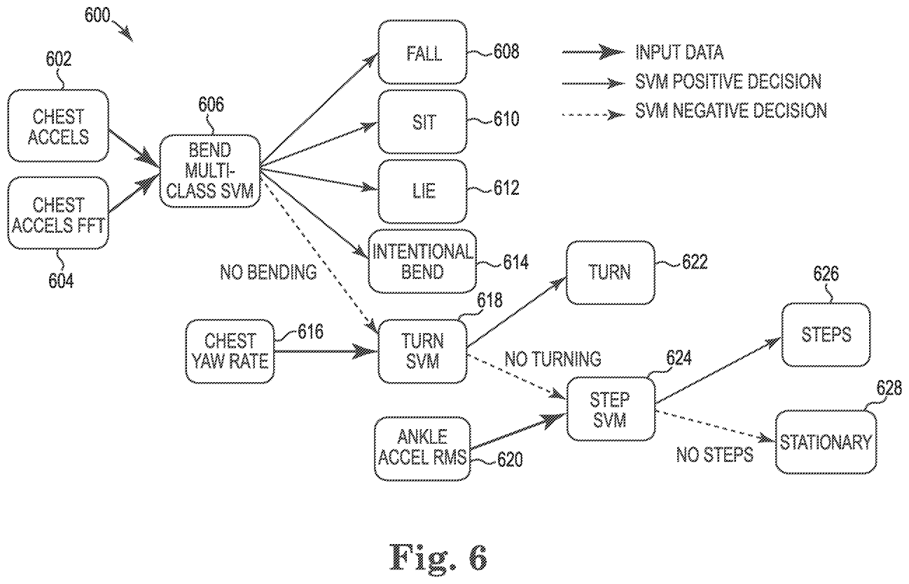

In addition to the three-sensor configuration shown in FIG. 4, other configurations with additional sensing devices 102 (e.g., a total of four or more sensing devices 102) may be used in order to improve the accuracy of measuring the subject's response to a near-fall event. FIG. 5 is a schematic diagram illustrating a wearable five-sensor configuration of sensing devices 102(1)-102(5) for a human subject according to an example. As shown in FIG. 5, five sensing devices 102(1)-102(5) are positioned on the subject. One of the sensing devices 102(2) is positioned just above the ankle on the subject's right leg. A second one of the sensing devices 102(3) is positioned just above the ankle on the subject's left leg. A third one of the sensing devices 102(1) is positioned on the chest of the subject. A fourth one of the sensing devices 102(4) is positioned on the thigh of the subject's right leg. A fifth one of the sensing devices 102(5) is positioned on the thigh of the subject's left leg. The additional sensing devices 102(4) and 102(5) on the thighs of the subject measure upper leg motion and may help to improve the accuracy of step length estimates. A four-sensor configuration may be configured in manner similar to that shown in FIG. 4, but a sensing device 102 may be placed on a single thigh rather than both thighs of the subject, for example. In an example, the sensing devices 102 do not need to be precisely located or precisely aligned. Examples disclosed herein may automatically calibrate and compensate the sensor signals to account for the unknown alignment of the sensing devices 102.

The wearable sensing devices 102 enable the performance of the following functions by computing device 104: (1) Automatic recognition of activities of a human subject that may include walking, sitting, lying down, bending, and other daily living activities involving motions of the chest and legs; (2) automatic recognition of near-fall events, which may include postural instability followed by balancing to recover from the near-fall event; (3) automatic estimation of the orientations and alignments of the sensing devices 102 affixed to the body, and estimates of the real-time bias in sensor signals of sensing devices 102; (4) automatic estimation of the real-time tilt angles of chest and leg segments during subject motion; (5) automatic estimation of reaction time, step lengths, and number of balancing steps in recovering from a near-fall event; and (6) automatic estimation of real-time chest anterior-posterior acceleration and velocity, which may be metrics indicating severity of a fall event.

### II. Automatic Near-Fall Event Recognition

In some examples, computing device 104 identifies the occurrence of near fall events based on sensor data received from sensing devices 102. While daily living includes a large number of various activities, a near-fall event may be characterized by specific types of changes in chest tilt angle followed by balancing steps to recover from the near-fall. This may involve a detection of bending (e.g., change in chest tilt angle), its characterization, detection of walking (e.g., balancing recovery steps), and estimation of step lengths. All of these data analysis steps may be performed using the sensor data from the sensing devices 102 on the chest and on the lower legs, for example, of the subject.

In some examples, computing device 104 may perform a near-fall detection method that is based on an activity classification decision tree. FIG. 6 is a diagram illustrating an activity classification decision tree 600 using support vector machines (SVMs) for detection of a fall or near-fall event followed by balancing steps according to an example. Some examples disclosed herein use a combination of both domain knowledge and machine learning methods to obtain a more tractable and reliable activity classification system. Note that the use of three IMUs 208, each with a 3-axis accelerometer and a 3-axis gyroscope, means the availability of a total of 18 sensors signals for real-time analysis. A brute force direct application of machine learning by supplying all 18 signals for training a multi-class SVM algorithm may lead to a complex system involving a tremendous amount of data for good training. This is because many (and even all) of the 18 signals may change in response to fall, walk, and many other daily living activities. However, not all of these signals play a primary role in characterizing a particular activity. By combining domain knowledge, just one or two of the 18 sensor signals may be used at each binary classification stage in the decision tree, highly reducing the size and complexity of the overall method. This enables the machine learning method to be trained using just minutes of data, instead of involving many days of representative daily living data sets for training.

A multi-class SVM 606 that utilizes chest accelerometers to identify bending activities is a first step in the decision tree shown in FIG. 6. Two-axis accelerations (anteroposterior and superioinferior) 602 and their windowed Fast Fourier Transform (FFT) 604 may be utilized to differentiate between non-bending activities and various activities that involve some bending. The FFT signals 604 of the chest accelerations play a role in differentiating between four common actions involving bending, namely Fall 608, Sit 610, Lie 612, and Intentional Bending 614. Just the 2-axis chest acceleration signals 602 from the sensing device 102 on the subject's chest and their FFTs 604 may be adequate to reliably differentiate between these sets of activities, and in some examples, none of the gyroscope signals of the sensing device 102 on the subject's chest or any of the signals from the sensing devices 102 on the legs are used for this differentiation.

If the activity is classified as not involving bending, then it is further classified as “Turning” 622, “Stepping” 626, or “Stationary” 628. The chest gyroscope yaw rate signal 616 by itself may be adequate for turn SVM 618 to classify an activity as involving “turning” 622 or “not turning.” If the activity does not involve turning, then the root mean square (RMS) of the accelerations signals 620 on the two legs may be used by step SVM 624 to detect whether there is an occurrence of “steps” 626 (e.g., walking, including the use of balancing steps after a near-fall) or if the subject is “stationary” 628. If there is an occurrence of steps after detection of a “near-fall” event, then the method for step length estimation may be triggered.

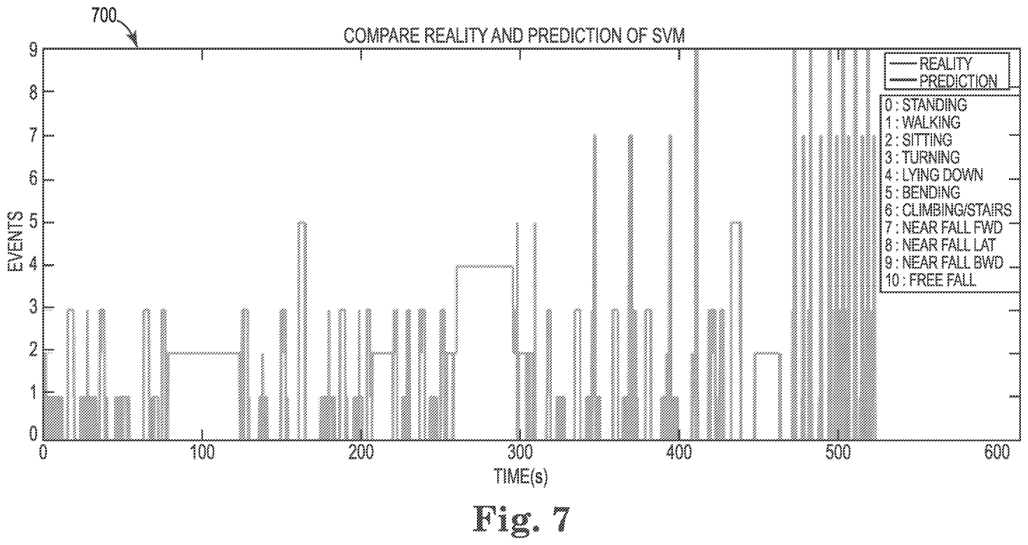

FIG. 7 is a diagram illustrating a graph 700 of results from approximately nine minutes of random activities by a human subject instrumented with three IMUs 208 (one each on the chest and two legs) according to an example. The activities involved were classified using techniques disclosed herein into one of ten categories at each instant in time. FIG. 7 shows both the actual activity as noted by a human referee and the activity recognized by an SVM decision tree. It can be seen that the recognition and the actual activities are largely identical, with the two curves almost on top of each other in the data. A discrepancy between the two curves occurs only at transients when one activity changes into another. The automatic recognition method used in some examples may be slightly delayed (e.g., by a fraction of a second) in guessing the new activity compared to the occurrence of the real activity.

In some examples, activity classification may be performed based on training transfer functions between appropriate sensor signals. The training process may use non-causal Wiener Filter solutions, and the selection of appropriate signals may be done through comparing the norms of the time-varying signals to determine the most relevant signals to each activity to be classified.

Some examples use IMUs 208 placed, for example, on the lower legs and chest, to classify a variety of common activities occurring throughout the day such as: walking, sitting, standing, turning, bending, trips, and near-falls (stumbles). Monitoring these activities can help characterize the postural stability of the subject and can also be useful for a number of other health applications. In order to properly classify these activities using three IMUs 208, deep learning techniques may be used.

Some examples are directed to an activity recognition machine learning method that uses a nonlinear observer to preprocess the data from IMUs 208. A nonlinear observer estimates body segment tilt angles and sensor bias parameters. In some examples, the estimates of the observer are used as the training data of a machine learning algorithm for activity recognition, instead of raw sensor data for this objective. In some examples, the activity recognition method is a deep learning-based activity recognition architecture, such as a convolutional neural network long short-term memory network (CNN-LSTM).

Some machine learning methods like SVM, k-Nearest Neighbors (KNN), and Vanilla Neural Networks (Vanilla NNs) are applicable to a wide range of problems, but may involve a lot of data engineering and preprocessing of the raw data before training the model to perform well. Furthermore, some of these methods may not be suitable for time series data and sequences since these data types may include lags and varied lengths. For example, in natural language processing, saying the same word could be done at different speeds. Therefore, ordinary machine learning methods may involve a tremendous amount of data to have all kinds of examples of the sample to perform well with time series data.

Recurrent Neural Networks (RNNs) have been introduced to overcome the problems of ordinary NNs with time series by getting feedback from the previous inputs unlike vanilla feedforward neural networks. This enables RNNs to perform better than vanilla neural networks in identifying and processing time series data like text and speech recognition.

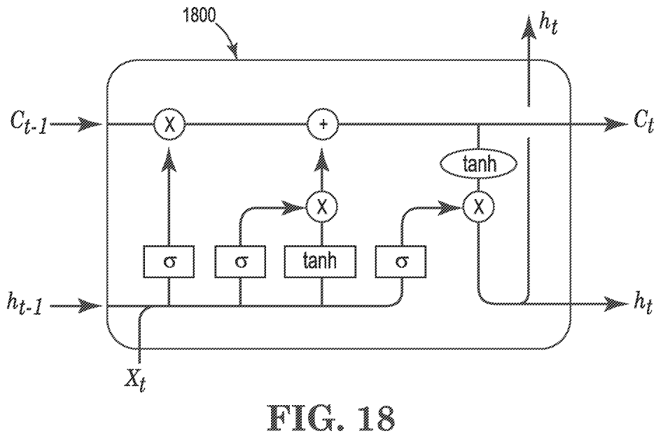

The vanishing gradient problem and insensitivity to gaps in the sequences of data in RNNs led to an upgraded version of RNNs called Long Short-Term Memory (LSTM) cells. FIG. 18 is a diagram illustrating an LSTM cell 1800 according to an example. In FIG. 18, t represents the time step, Cₜ is the cell state vector, hₜ is the hidden state vector, σ is the sigmoid activation function, and tan h is the hyperbolic tangent activation function. LSTM cell 1800 includes an input gate, an output gate, and a forget gate. The advantage of this cell 1800 over RNNs, hidden Markov models, and other sequence learning methods, is that it recalls values over arbitrary time intervals while the three gates adjust the data stream into and out of the cell.

An example CNN-LSTM includes two one-dimensional (1D) convolutional layers with 64 filters (size of window) and kernel size of 3 with ReLu activation function. To reduce the chance of overfitting, a dropout layer with 50% probability may be used. The CNN-LSTM may include a maxpooling layer with a size of two and a flatten layer to filter the features of the data. Other parameters may be set as defaults. The extracted features by the CNN are fed to a LSTM layer with 100 units and default activation function of hyperbolic tangent and recurrent sigmoid.

Since LSTM units need a time series of inputs to predict the outputs, the entire CNN model may be wrapped with a TimeDistributed layer to allow the CNN model to read multiple time sequences of data at the same time. A standard approach for TimeDistributed layers is to split input data to multiple windows for the CNN model. The data is, therefore, reshaped to 4 sequences of 50 time steps for each time window of 200 time steps (200*10 ms=2000 ms=2 sec).

Finally, another dropout layer and two Fully Connected (FC) layers may be added. The dropout layer has a 50% probability and the first dense layer has 100 units with ReLu activation. The final layer, which classifies the data to the final activities, is a dense layer with Softmax activation function with 9 nodes to complete the network.

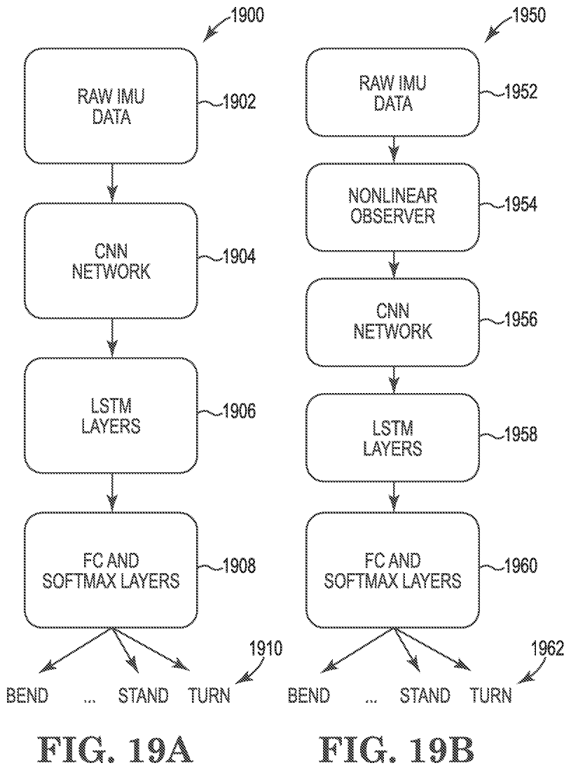

FIGS. 19A and 19B are block diagrams illustrating activity recognition methods using a CNN-LSTM according to an example. Method 1900 shown in FIG. 19A uses only raw IMU data, and method 1950 shown in FIG. 19B uses a nonlinear observer to estimate body segment tilt angles based on raw IMU data. In an example, the raw IMU data at 1902 and 1952 is generated from three IMU devices on the body, which each yields six signals (i.e., three acceleration signals and three angular rates), so 18 raw signals in total.

To evaluate the effect of the observer on the performance of the CNN-LSTM network in classifying activities, the performance of the two methods 1900 and 1950 is compared. In method 1900, all 18 raw IMU data signals are provided to the CNN network 1904 without the nonlinear observer layer 1954. In method 1950, the nonlinear observer layer 1950 is added to the method to estimate the tilt angle signal of each IMU. The nonlinear observer layer 1954 extracts three tilt angle signals from the raw IMU data, and only these three estimated tilt signals are then input to the CNN network 1956. The raw IMU sensor signals are not provided to the CNN network 1956 in method 1950. In method 1900, the raw IMU data 1902 is processed by the CNN network 1904, LSTM layers 1906, and FC and Softmax layers 1908 to generate activity classifications 1910 (e.g., bend, stand, turn, etc.). In method 1950, the tilt angles from nonlinear observer 1954 are processed by the CNN network 1956, LSTM layers 1958, and FC and Softmax layers 1960 to generate activity classifications 1962 (e.g., bend, stand, turn, etc.). The hyper parameters of the networks in methods 1900 and 1950 remain the same for both networks to have a fair experiment with similar situations.

To test and compare methods 1900 and 1950, three IMUs 208 were used, each including a 32-Bit ARM Cortex M4F processor, and a measurement IC with a 6-axis combo accelerometer and gyroscope. The sampling rate was chosen as 100 Hz, and the data was streamed wirelessly to a phone or computer via Bluetooth while also being stored on a 64 Mb on-board memory. The ground-truth measurement device was an OptiTrack infra-red motion capture system that is able to track passive and active markers with 120 Hz sampling rate in 3D space with sub-millimeter accuracies.

The estimates from the nonlinear observer 1954 were compared with the OptiTrack measurements, and the observer results match the camera measurements very well. The mean angle error of the observer was 3.59% and the maximum error was 10.98%.

A dataset was collected from the three IMUs 208 attached to the body, one on each shank and one on the chest, while the subject was doing nine different activities of bend, sit-stand, near-fall forward, near-fall backward, n-fall lateral, lie down supine, lie down sideways, stand, and turn. The original data was sampled at 100 Hz. The sensor signals were preprocessed by sampling sliding windows of two seconds with a 50% overlap (200 readings/window). The final dataset consists of 8783 samples including 7026 training samples and 1757 test samples.

A confusion matrix of the first method 1900 with only raw data as the input of the CNN-LSTM network was generated. A confusion matrix for the second method 1950 using the observer estimates as the inputs was also generated. These matrices show the number of predicted labels for each sample compared to the true labels. Based on these results, the average accuracy of the first method 1900 was about 96.47%, while with the help of the nonlinear observer in the second method 1950, it increases to 99.81%. A reason for this difference is that the raw data includes noise, biases and it is generally harder to reliably find patterns in it due to its larger volume (18 signals vs. 3 signals). A deep learning algorithm needs more training data to find patterns in the raw data, since it has both a higher number of signals and more noise and bias errors.

An activity recognition plot was generated for methods 1900 and 1950 to show the overall activity recognition performance of the CNN-LSTM network with the raw and observer-estimated data compared to each other and with the true labels. The CNN-LSTM performed better and trained faster with the observer-processed data. The generated plot shows that most of the incorrect labels of the network trained on the raw data are related to near-fall events due to similarities between these activities, sit-stands and bends.

This disclosure sets forth a nonlinear switched-gain observer based on measurements from IMUs 208 worn on leg segments and chest in order to estimate orientation of body segments for the purpose of preprocessing data for a deep learning method of activity recognition. The observer estimates the tilt angles and measurement bias of each IMU 208 using a switched-gain strategy for three different working regions in each of which the output nonlinear function is monotonic. Using this observer, two different methods 1900 and 1950 have been presented to compare the effect of the observer on the activity recognition method. In the first method 1900, the 18 signals of raw data measured by the IMU devices was fed directly to the CNN-LSTM network. In the second method 1950, the nonlinear observer was applied to the raw measurements to estimate tilt angles of each sensor. These estimated angles were then used as the training data of the network.

The developed nonlinear switched-gain observer turns out to be a powerful tool in estimation of the orientation angles of IMU sensors and has the potential to be applied wherever IMUs are employed in applications like navigation, activity recognition, and stabilization.

### III. Estimation of Response Variables During Near-Fall Event

In some examples, computing device 104 estimates several variables during a near-fall event based on sensor data received from sensing devices 102. In some examples, computing device 104 estimates the following variables in real-time.

(1) The gyroscope and accelerometer signals from the sensing devices 102 may have bias errors and these bias values may change slowly over time. Even small bias values can cause large errors in signals estimated by integration. Hence, in some examples, these bias values are estimated in real-time so that they can be subtracted before integration.

(2) The sensors affixed to the body may have orientation errors with respect to inertial (i.e., global) axes. The users may not be able to perfectly align the sensing devices 102 while fixing them on the body. Hence, automatic estimation of the orientations of the sensor axes may be performed.

(3) Automatic estimation of the real-time tilt angles of chest, upper leg and lower leg segments during subject motion may be performed. These tilt angles enable estimation of horizontal and vertical components of accelerations, and enable calculation of step lengths based on tilt angles and limb lengths.

(4) Estimation of the start time and end time of a step during walking/balancing may be performed.

(5) Automatic estimation of reaction time, step lengths, and number of balancing steps in recovering from a near-fall event may be performed.

### IV. Step Length Estimation

### A. Introduction

In some examples, computing device 104 estimates step length information based on sensor data received from sensing devices 102. Some examples perform step length estimation using either one IMU 208 on each shank, or one IMU 208 on each shank and one IMU 208 on each thigh of a human subject. An observer design problem that involves estimating shank angle, thigh angle and bias parameters of the initial sensors in order to estimate step lengths is formulated. A nonlinear observer is designed using Lyapunov analysis to solve the formulated problem and utilizes a linear matrix inequality (LMI) to find a stabilizing observer gain. In some examples, switched gains are used (e.g., one gain for each piecewise monotonic region of the nonlinear output function) to help ensure global stability of the observer over the entire operating region for thigh and shank angles. Experimental results are presented on the performance of the nonlinear observer and compared with reference measurements from an infrared camera capture system.

IMU sensors may suffer from measurement bias and drifts. Hence, estimation of displacement and angular motion using integration of IMU sensor signals tend to easily drift over time. A problem with some approaches is that they make several assumptions on the initial and boundary conditions and utilize gait constraints, which are not uniformly correct for different persons and situations. In addition, other types of movements besides gait are typically ignored or not well characterized in the literature, but are likely quite relevant in a variety of disease processes. For estimation of tilt angle and measurement bias of IMUs, examples disclosed herein use nonlinear models and estimates.

This disclosure develops a nonlinear observer for the step length estimation problem based on solving linear matrix inequalities. The observer is designed and proved to be stable for a system with linear dynamics and non-monotonic nonlinear outputs. An objective of this nonlinear observer is to estimate the real-time vertical in-plane angle of the IMU 208 relative to the gravity vector as well as the accelerometer and gyroscope biases. The real-time angle is used in finding the orientation of the body limb and also for transforming the accelerations to the inertial frame. This information may be utilized in three different approaches for estimation of human step length. The first method integrates the linear accelerations of shank sensors and gives the velocity and displacement of the sensors in space and hence the step lengths by assuming zero initial conditions at the start moment of each step. The second method estimates the orientations of multiple sensors and takes advantage of the geometry of the body to estimate step lengths. The third method assumes that each leg has a stance and swing phase during walking, with the thigh angular trajectory being symmetric between the two legs. In the first method, only two sensing devices 102 are used (e.g., one for each shank), while the second, angle-based method uses four sensing devices 102 for finding the orientations of both shanks and thighs of a subject. In the third method, three sensing devices 102 are used (e.g., one for each shank, and one for one thigh). The measurements are done using IMUs 208 and the data are logged and transferred by wireless connection to a smartphone or computer. The accuracy of the nonlinear observer and these methods are compared to an infrared motion capture camera system. The results show very good reliability of both methods, but the angle-based method presents less errors in terms of step length estimation.

Stochastic estimation filters such as a Kalman Filter, an unscented Kalman Filter or an extended Kalman Filter may also be equivalently utilized instead of a nonlinear observer for these estimation tasks involving estimation of shank and thigh angles, sensor bias values and step length estimation by integration-based or angle-based methods.

The following description is organized as follows. In Section B, the problem formulation is presented. In Section C, the nonlinear observer design algorithm is discussed. In Section D, three different step length estimation methods are described. In Section E, experimental results for validating the accuracy of the nonlinear observer and the step estimation methods are presented. Finally, section F presents conclusions.

### B. Problem Formulation

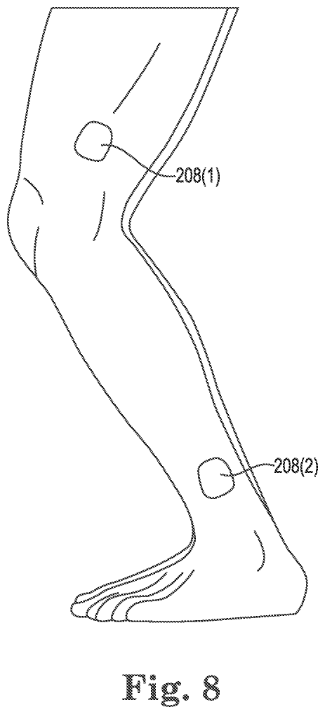

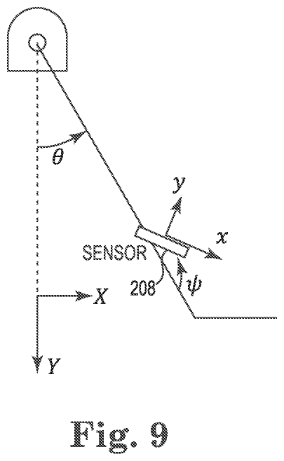

The following description assumes the use of either one IMU 208 on the shank or two IMUs 208, one each on the shank and thigh, as shown in FIG. 8. FIG. 8 is a diagram illustrating a leg of a human subject with a first IMU 208(2) positioned on a shank of the leg and a second IMU 208(1) positioned on the thigh of the leg according to an example. FIG. 9 is a diagram illustrating the axes of the IMU 208 and the attachment of it to a body limb segment according to an example. θ is the real-time absolute (inertial) angle of the segment and ψ is the unknown orientation angle of the sensor on the limb (due to misalignment). Although ψ is unknown, it does not change with time, while the segment angle θ may continuously change with time. The IMU 208 fixed axes are defined as (x,y) and the inertial frame fixed axes are (X,Y) as shown in FIG. 9. The variables aₓ, a_{y}, a_{X} and a_{Y} are the true accelerations along these axes, respectively.

The IMU orientation on each shank is θ+ψ, where θ is the real-time angle of the shank and ψ is the additional mounting angle of the IMU on the shank. Let ϕ=θ+ψ. Then the measurements of the accelerometers can be described by (outputs y₁ and y₂):

y₂=a_{ym}=a_{y}−g cos(ϕ)+b_{ay} (1)

y₁=aₓₘ=aₓ−g sin(ϕ)+bₐₓ (2)

The gyroscope signal measured by the IMU is:

a=ω_{gz}=(θ+ψ)_{measured}=θ+b_{gz} (3)

Here bₐₓ, b_{ay} and b_{gz} are the unknown bias values of the accelerometer and the gyroscope measurements, respectively, and are assumed to be constant. The relationship of the sensor measured signals to the inertial accelerations is given by:

a_{X}=aₓ cos(θ+ψ)+a_{y} sin(θ+ψ) (4)

a_{Y}=a_{y} cos(θ+ψ)−aₓ sin(θ+ψ) (5)

Then the overall dynamics of the IMU system on each lower leg can be described by the following equations, where ω_{gz}=y_{z}:

ϕ=ω_{gz}+b_{gz}

bₐₓ=0

b_{ay}=0

b_{gz}=0 (6)

In matrix form, the system dynamics are:

$$\frac{d}{d \text{?}} \{ \begin{matrix}ϕ \\ {b}_{ax} \\ b \text{?} \\ b \text{?}\end{matrix} ) \text{?} [ \begin{matrix}0 & 0 & 0 & 1 \\ 0 & 0 & 0 & 0 \\ 0 & 0 & 0 & 0 \\ 0 & 0 & 0 & 0\end{matrix} ] \{ \begin{matrix}ϕ \\ b \text{?} \\ b \text{?} \\ b \text{?}\end{matrix} \} + [ \begin{matrix}1 \\ 0 \\ 0 \\ 0\end{matrix} ] \text{?} \qquad ( 7 )$$

$$y \text{?} \{ \begin{matrix}{y}_{1} \\ y \text{?}\end{matrix} \} \text{?} h ( Ex ) + Cx + {DA}_{input} \qquad ( 8 )$$

$$where$$

$$E = [ \begin{matrix}1 & 0 & 0 & 0\end{matrix} ] \\ C = [ \begin{matrix}0 & 1 & 0 & 0 \\ 0 & 0 & 1 & 0\end{matrix} ] \\ {\text{?}}_{input} = \{ \begin{matrix}\text{?} \\ \text{?}\end{matrix} \} \\ h ( Ex ) = \{ \begin{matrix}{h}_{1} ( Ex ) \\ {h}_{2} ( Ex )\end{matrix} \} = \{ \begin{matrix}- g sin \text{?} \\ - g cos ϕ\end{matrix} \} \qquad ( 9 )$$

$$\text{?} \text{indicates text missing or illegible when filed}$$

In general system notation form, the system model is

$$\frac{d}{dt} x \text{?} Ax + \text{?} \qquad ( 10 )$$

$$\text{?} \text{?} Cx \text{?} h ( Ex ) \text{?} {DA}_{input} \qquad ( 11 )$$

$$\text{?} \text{indicates text missing or illegible when filed}$$

### C. Nonlinear Observer Design

Let the observer be given as

$$\frac{d}{dt} \text{?} \text{?} A \text{?} + \text{?} + L \{ Cx + h ( Ex ) + {DA}_{input} - C \text{?} - h ( E \text{?} ) \} \qquad ( 12 )$$

$$\text{?} \text{indicates text missing or illegible when filed}$$

Let the estimation error be:

\tilde{x}=x−\hat{x} (13)

Then the estimation error dynamics are given by:

\hat{x}=\hat{x}−\hat{x}=A\hat{x}−Lh(Ex)+Lh(E\hat{x})−LDAᵢₙₚᵤₜ

or

\hat{x}=(A−LC)\hat{x}−L\tilde{h}(Ex,E\hat{x})−LDAᵢₙₚᵤₜ (14)

where \tilde{h}(Ex,E\hat{x})=h(Ex)−h(E\hat{x}).

### Lemma 1:

The nonlinear difference function \tilde{h}(Ex,E\hat{x}) satisfies the quadratic inequality

$${V}_{1} = [ \begin{matrix}\text{?} & \text{?} ( Ex , E \text{?} ) \text{?} ] \text{?} [ \text{?} ( {Ex , Ex \text{?}}^{\text{?}} ) ]\end{matrix} \leq 0 \qquad ( 15 )$$

$$where \\ \text{?} \text{?} [ \begin{matrix}\frac{{E}^{T} {M}^{T} NE + {E}^{T} {N}^{T} ME}{2} & - \frac{{E}^{T} {M}^{T} + {E}^{T} {N}^{T}}{2} \\ - \frac{ME + NE}{2} & I\end{matrix} ]$$

$$\text{?} \text{indicates text missing or illegible when filed}$$

where

$$M \text{?} [ \begin{matrix}{M}_{1} & \text{?} \\ 0 & {M}_{2}\end{matrix} ] and N \text{?} [ \begin{matrix}{N}_{1} & \text{?} \\ 0 & {N}_{2}\end{matrix} ]$$

$$\text{?} \text{indicates text missing or illegible when filed}$$

are diagonal matrices containing the lower and upper bounds on the partial derivates of h₁(Ex)=g sin ϕ and h₂(Ex)=−g cos ϕ respectively.

### Proof

Lemma 1 can be proved using the differential mean value theorem.

### Theorem 1:

If the acceleration input Aᵢₙₚᵤₜ is zero, then the errors in the estimates of the angle and the bias parameters will converge globally exponentially to zero, if the observer gain is selected so that the following LMI-equivalent inequality

$$S < 0 \qquad ( 16 )$$

$$with \\ \text{?} = ( A \text{?} LC ) \text{?} F + F ( A \text{?} LC ) \text{?} \frac{E \text{?} M \text{?} NE + E \text{?} N \text{?} ME}{\text{?}} + \text{?} F \\ \text{?} = \text{?} FL \text{?} \frac{E \text{?} M \text{?} \text{?} E \text{?} N \text{?}}{\text{?}} \\ \text{?} = \text{?} \text{?} \text{?} \text{?} \frac{ME + NE}{2} \\ \text{?} = - \text{?}$$

$$\text{?} \text{indicates text missing or illegible when filed}$$

yields a positive definite solution P>0 and the observer gain L.

### Proof

Consider the Lyapunov function candidate

V−\tilde{x}^{T}P\tilde{x} (17)

with P>0 being a positive definite matrix. Then

$$\text{?} = \text{?} P \text{?} + \text{?} P \text{?} = \text{?} [ {( A - LC )}^{T} P + P ( A - LC ) ] \text{?} - \text{?} PL \text{?} ( Ex , E \text{?} ) - \\ \text{?} ( Ex , E \text{?} ) L \text{?} P \text{?} - \text{?} {PLDA}_{input} - A \text{?} D \text{?} L \text{?} P \text{?} \qquad ( 18 )$$

$$\text{?} \text{indicates text missing or illegible when filed}$$

If Aᵢₙₚᵤₜ−0, then

$$\text{?} = \text{?} [ ( A - \text{?} C ) \text{?} P + P ( A - \text{?} C ) ] \text{?} - \text{?} PL \text{?} ( Ex , E \text{?} ) \text{?} \text{?} ( Ex , E \text{?} ) {L}^{T} P \text{?} \qquad ( 19 )$$

$$Or$$

$$\text{?} = [ \begin{matrix}\text{?} & \text{?} ( Ex , E \text{?} ) \text{?} ] [ \begin{matrix}{( A - LC )}^{T} P + P ( A - LC ) & - PL \\ - {L}^{T} P & 0\end{matrix} ] [ \begin{matrix}\text{?} \\ \text{?} ( Ex , E \text{?} )\end{matrix} ]\end{matrix}$$

$$\text{?} \text{indicates text missing or illegible when filed}$$

### Using the S-Procedure Lemma,

\dot{V}<0 if and only if there exists ϵ>0 such that \dot{V}≤eV₁.
Hence, \dot{V}<0 if and only if

$$[ \begin{matrix}\text{?} & \text{?} {( Ex , E \text{?} )}^{T} ] [ \begin{matrix}{( A - LC )}^{T} P + P ( A - LC ) & - PL \\ - {L}^{T} P & \text{?}\end{matrix}\end{matrix} ] [ \begin{matrix}\text{?} \\ \text{?} ( Ex , E \text{?} )\end{matrix} ] \leq \text{?} \begin{matrix}[ \text{?} & \text{?} {( Ex , E \text{?} )}^{T} ]\end{matrix} [ \begin{matrix}\frac{E \text{?} M \text{?} NE + E \text{?} N \text{?} ME}{2} & - \frac{E \text{?} M \text{?} + E \text{?} N \text{?}}{2} \\ - \frac{ME + NE}{2} & \text{?}\end{matrix} ] ⌈ \begin{matrix}\text{?} \\ \text{?} ( Ex , E \text{?} )\end{matrix} ⌉ \qquad ( 20 )$$

$$or$$

$$S < 0$$

$$where$$

$$\text{?} = \frac{\text{?} A \text{?} C \text{?} F}{\text{?}} + \frac{F}{\text{?}} \text{?} A \text{?} \text{?} C \text{?} \text{?} \frac{E \text{?} M \text{?} NE + E \text{?} N \text{?} ME}{\text{?}} \\ \text{?} = - PL / \text{?} + \frac{E \text{?} M \text{?} + E \text{?} N \text{?}}{2} \\ \text{?} = - \frac{L \text{?} P}{\text{?}} + \frac{ME + NE}{2} \\ \text{?} = - \text{?}$$

$$\text{?} \text{indicates text missing or illegible when filed}$$

Replacing by a new positive definite matrix P₁=P/e, and adding a σP term to the (1,1) entry for a guaranteed exponential convergence rate of σ/2, the result follows.

Low-pass filtered versions of y₁ and y₂ can be used in the observer so that the dynamic components are removed and Aᵢₙₚᵤₜ can indeed be negligible. In other words,

y₁=a_{ym}=a_{y}−g cos(θ+ψ)+b_{y}

y₁=aₓₘ=aₓ−g sin(θ+ψ)+bₓ

while

y_{2_low_pass}=−g cos(θ+ψ)+b_{y}

y_{1_low_pass}=−g sin(θ+ψ)+bₓ

### Hence,

$$\{ \begin{matrix}\text{?} \\ \text{?}\end{matrix} \} = Cx + h ( Ex ) ,$$

$$\text{?} \text{indicates text missing or illegible when filed}$$

instead of

$$\{ \begin{matrix}\text{?} \\ \text{?}\end{matrix} \} \text{?} Cx + h ( Ex ) + {DA}_{input} ,$$

$$\text{?} \text{indicates text missing or illegible when filed}$$

and thus

$${A}_{input} = \{ \begin{matrix}\text{?} \\ \text{?}\end{matrix} \} = \text{?} .$$

$$\text{?} \text{indicates text missing or illegible when filed}$$

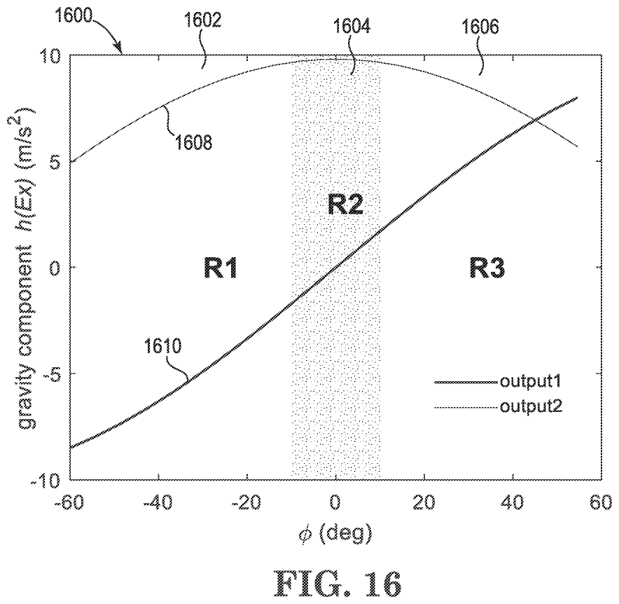

FIG. 16 is a diagram illustrating a graph 1600 of two outputs 1608 and 1610 with assumed constant-gain operating regions 1602, 1604, and 1606 of the nonlinear observer according to an example. As shown in FIG. 16, the output function y₂, i.e., h₂(Ex), is non-monotonic. Thus LMI equation (16) is not feasible over the entire operating range of orientation angles of ϕ from −60 to 60 degrees. To solve this issue, feasible solutions may be found for each monotonic region in the operating regions 1602, 1604, and 1606, shown by R1, R2 and R3, respectively. In the first region 1602 and the third region 1606, the function h has a monotonic response in both channels 1608 and 1610. While in the second region 1604, only the first output 1610 is monotonic. Hence for the regions 1602 and 1606, the observer can be designed using both outputs 1608 and 1610, whereas in the second region 1604, the observer only uses the first output 1610.

A hybrid nonlinear observer that uses multiple constant-gain stable regions and switches between these stable gains with adequate dwell time after each switching is globally asymptotically stable. A switched-gain hybrid observer with three constant gains may be designed, and the LMI of Equation (16) may be solved to obtain the three constant observer gains.

### D. Step Length Estimation

In this part three different methods are considered for estimating step lengths using configurations of two, three, and four sensors, respectively, on the two legs of the subject. For the first method, it is assumed that there are only two IMUs 208 (one attached to each shank) and an integrator-based method is used to estimate step length. For the second method, we assume four sensors in all—each leg has two IMUs 208, one IMU 208 on each shank and one IMU 208 on each thigh, and the estimated angles of both the shank and the thigh are used to find the step length. For the third method, we assume there is one sensor attached to each shank and a third sensor is attached to one of the thighs.

### Integrator-Based Estimation

If there is only one IMU 208 on each shank, one can find the step lengths based on the double-integration of the linear acceleration of the foot in the inertial frame. For this, we need to find the real-time orientation of the sensor relative to the inertial frame and then transfer the corrected/unbiased accelerations (found from nonlinear observer) using the following direction cosine matrix (DCM) to the inertial frame. Subsequently, the gravity component from the transformed accelerations are removed. Then the resulting dynamic accelerations are double integrated from the end of one step to the end of the next step. The DCM is given by:

$$R ( ϕ ) = [ \begin{matrix}cos ( ϕ ) & - sin ( ϕ ) \\ sin ( ϕ ) & cos ( ϕ )\end{matrix} ] \qquad ( 21 )$$

With the relationship between inertial and sensor accelerations given by:

$$\{ \begin{matrix}\text{?} \\ \text{?}\end{matrix} \} \text{?} R ( \text{?} ) \{ \begin{matrix}\text{?} \\ \text{?}\end{matrix} \}$$

$$\text{?} \text{indicates text missing or illegible when filed}$$

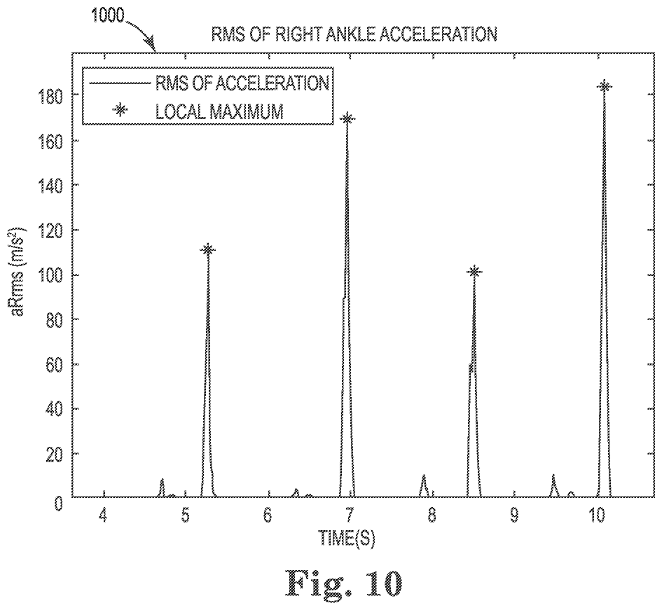

The end of each is step is found based on the high peaks in the RMS signal of each accelerometer. FIG. 10 is a diagram illustrating a graph 1000 of an example RMS signal of a right foot acceleration and its local maximum that are used to identify the heel-strikes of each step according to an example. FIG. 10 shows that the end of each step has a clear local maximum in the RMS signal, which is because of heel-strikes. The start of integration is found based on the assumption of step-by-step walking. This means that the beginning of each step coincides with the end of the previous step. Experimental data has shown that starting integration earlier than the real lift off does not degrade accuracy because the foot is stationary. On the other hand, the accuracy degrades very fast if the integration is started late. Good accuracy has been seen by assuming that foot lift off happens at the end of the previous step.

### Angle-Based Estimation

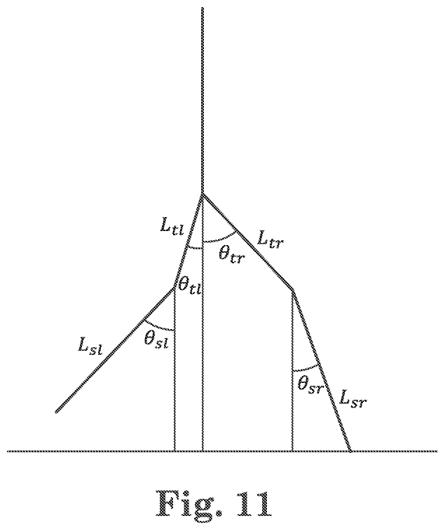

Another method is to estimate the step lengths from the geometry of the body during in-plane walking. FIG. 11 is a schematic diagram illustrating a model of the body and the angles of each limb during in-plane walking according to an example. In this method, four IMUs 208 are used, two IMUs 208 for each leg (one IMU 208 on each shank, and one IMU 208 on each thigh) to be able to estimate the angles of both shank and thigh in real-time. The angles of right shank, right thigh, left shank, and left thigh with vertical axis are shown as θₛᵣ, θₜᵣ, θₛₗ, and θₜₗ respectively. Also the length of right shank, right thigh, left shank, and left thigh are Lₛᵣ, Lₜᵣ, Lₛₗ, and Lₜₗ respectively. Based on FIG. 11, we can calculate the real-time relative position of the feet using the following equations and so find the step lengths at the end of each step. For example, assume that the right foot is on the ground and the left leg is swinging then we have the position of right foot relative to the left foot:

X_{pfl}=Lₛᵣ sin(θₛᵣ(t))+Lₛᵣ sin(θₛᵣ(t))+Lₛₗ sin(θₛₗ(t))+Lₜₗ sin(θₜₗ(t)) (22)

Y_{pfl}=Lₛₗ cos(θₛₗ(t))+Lₛₗ cos(θₛₗ(t))−Lₛᵣ cos(θₛᵣ(t))−Lₜᵣ cos(θₜᵣ(t)) (23)

The time of end of each step is found from the high peaks experienced in the RMS signal of each accelerometer. If it is assumed that we detect an end of step in the left foot, the step length is therefore computed as below:

Step length=Lₛᵣ sin(θₛᵣ(tₛₗ))+Lₜᵣ sin(θₜᵣ(tₛₗ))+Lₛₗ sin(θₛₗ(tₛₗ))+Lᵣₗ sin(θₜₗ(tₛₗ)) (24)

in which tₛₗ is the time at the end of left foot step.

### Polynomial Angle-Based Estimation

In the polynomial angle-based estimation method according to an example, two shank sensors and one thigh sensor are used. The angle of the thigh without a sensor may be estimated by a polynomial function of time, based on the angle of the thigh with a sensor and then using Equation (24) to estimate the step lengths from the geometry of in-plane walking.

Walking may be divided into the two main parts of swing and stance phases based on the angles of the thigh and shank. During the stance phase, the foot is on the ground and the angle of the shank and thigh are almost equal (shank and thigh are aligned). However, during the swing phase, the leg is swinging in the air and the angle of thigh diverges from that of the shank.

For the swing phase, the thigh angle may be estimated from a polynomial fitted to the swing phase angle of the other leg as described below:

(1) Estimate left thigh angle \hat{θ}ₜₗ using the nonlinear observer described in equation (12) since we have IMU on the left thigh.

(2) Fit a 4^{th} order polynomial to the estimated left thigh angle \hat{θ}ₜₗ using least squares:

\hat{θ}ₜₗ(t)=p₄t⁴+p₃t³+p₂t²+p₁t+p₀ (25)

in which t shows time, pᵢ are the polynomial coefficients found from a least squares solution, and θₜₗ is the estimated left thigh angle.

(3) Estimate the right thigh angle using the calculated polynomial by shifting the time relative to the start of the right footstep.

$$\text{?} ( t ) = \text{?} ( t - t \text{?} ) \text{?} + \text{?} ( t - t \text{?} ) \text{?} + \text{?} ( t - t \text{?} ) \text{?} + \text{?} ( t - t \text{?} ) + {p}_{0} \qquad ( 26 )$$

$$\text{?} \text{indicates text missing or illegible when filed}$$

in which tₛᵣ shows the start time of the right footstep.

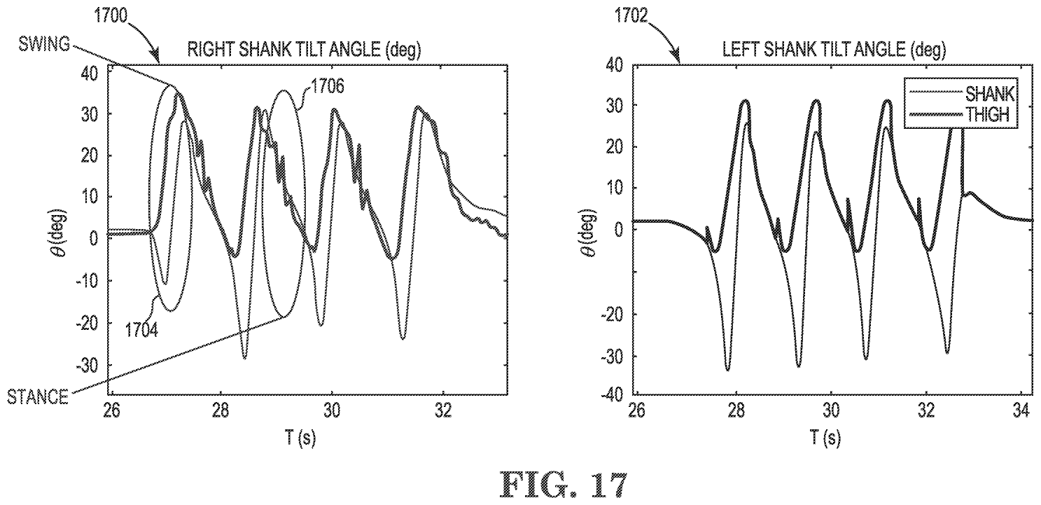

FIG. 17 is a diagram illustrating graphs 1700 and 1702 of shank and thigh tilt angles according to an example. FIG. 17 shows the angles of the shank and thigh of both legs during stance phase 1706 and swing phase 1704. The shank angles of both left and right legs, and the thigh angle of the left leg are estimated from the nonlinear observer. The thigh angle of the right leg is assumed to be equal to the shank angle during stance phase, however, during swing phase, it is estimated by polynomial fitted using least squares to the left leg thigh angle estimates. A comparison of such estimates with IR camera measurements has been made and shows that the right thigh angle can be successfully estimated using this method.

### E. Experimental Results

Inexpensive commercial IMUs 208 are used as the measurement sensors. Each device has a 32-Bit ARM Cortex M4F processor powered by high capacity CR2450 Lithium Coin Cell. The measurement sensor includes a three-axis accelerometer and a three-axis gyroscope. The data from the IMUs 208 is sampled at a 100 Hz frequency and stored on a flash memory, and also may be streamed through Bluetooth connectivity to a smartphone or computer.

An infrared (IR) motion tracking camera is used as an accurate reference sensor with which the estimated step lengths and angles from the nonlinear observer are compared. The setup includes a self-contained and factory calibrated camera and uses three cameras and built-in software to track passive markers attached to the body with a sub-millimeter accuracy. The sample rate of the camera system is 120 Hz.

The test consists of two parts of walking and stumbling in both forward and backward directions to observe the accuracy of the estimations in low and high acceleration motions. The term “stumbling” means simulation of near-fall activities. Stumbling was included in addition to walking in order to show that the methods disclosed herein work in the presence of disturbances and to prepare for other future studies. The first 30 seconds of the test is walking, and the rest of the test data consists of walking and stumbling.

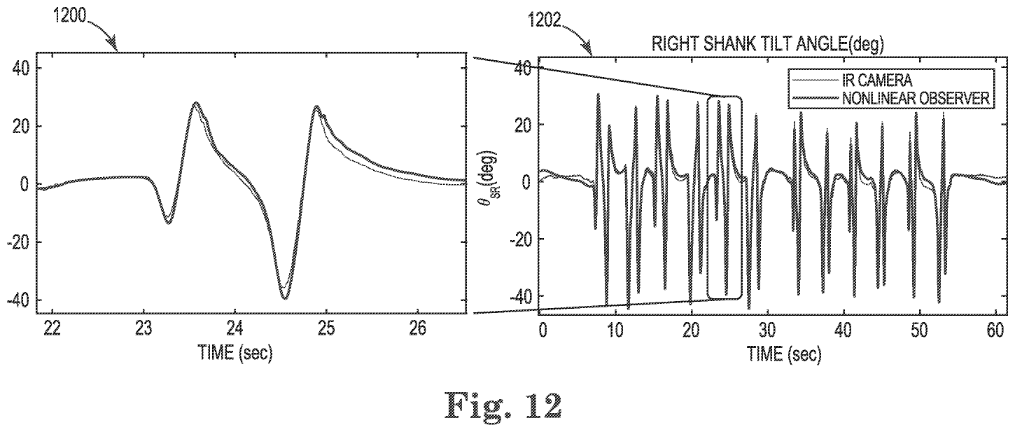

An optimum value of σ=0.45 for the convergence rate was found numerically to yield the minimum estimation error rate. We can compare the overall accuracy of the shank angle estimation of the nonlinear observer with the IR camera measurements with reference to FIG. 12. FIG. 12 is a diagram illustrating graphs 1200 and 1202 of IR camera measurements and shank tilt angle estimations of a nonlinear observer according to an example. It can be seen in FIG. 12 that the shank angle estimates track the IR camera angle estimates very closely.

To show the effect and the importance of the nonlinear observer on estimation of the step lengths, the direct double integration of raw IMU acceleration measurements were plotted to find foot displacement. Also, tilt angles from direct integration of gyro measurements were compared to IR camera measurements. The direct integration of gyro has a linear growing drift while double integration of accelerations results in an exponential growing position drift due to not having removed the bias and tilt angle errors in IMU measurements.

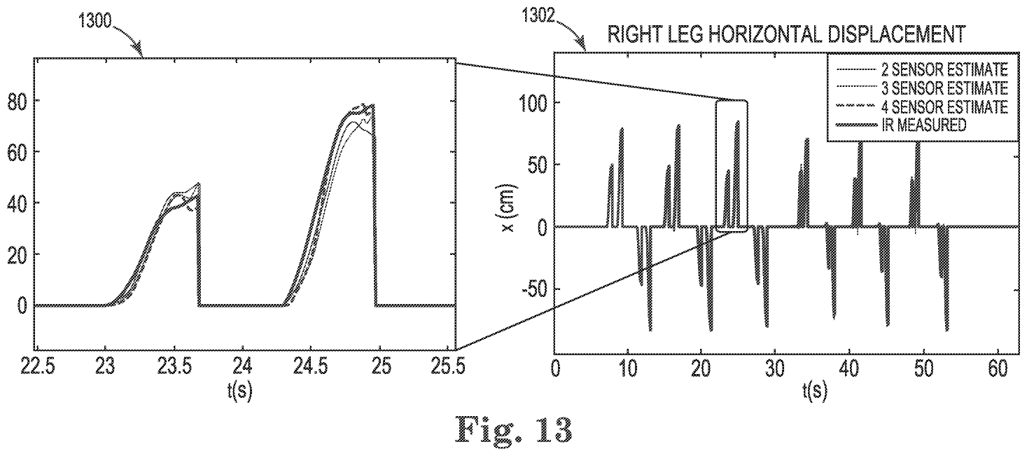

FIG. 13 is a diagram illustrating graphs 1300 and 1302 of a comparison of step length (e.g., horizontal displacement) estimation of the three observer-based methods (i.e., two-sensor integrator-based, four-sensor angle-based, and three-sensor polynomial angle-based) with an IR camera according to an example. Based on these results, the average accuracy of the two-sensor integrator-based method is 91.42%, for the three-sensor angle-based method is 94.64%, and for the four-sensor angle-based method is 95.67%. A reason for this difference is that the integrator-based method depends on the initial conditions and measurement biases with high sensitivity because of double integration, while the angle-based method uses the real-time estimation of the angles and is independent of initial conditions and less influenced by the bias errors. Also, a reason for the difference between four and three sensor methods is that the three sensor method relies on a polynomial for the estimation of right thigh angle, while the four sensor method estimates it by the nonlinear observer from direct measurement of all four IMUs.

The latter 30 seconds of the test includes the stumbling part of the test. In this part, we see more errors, especially in the integration mode. The average step length error in the second half of the test is 5.93% for the four-sensor angle-based method while it is 10.34% for the three sensor method and 26.32% for the integrator-based method. Again, the integration mode shows more sensitivity to the initial conditions and errors due to the impulses during stumbling. Also, the polynomials become less accurate due to differences between walking and stumbling.

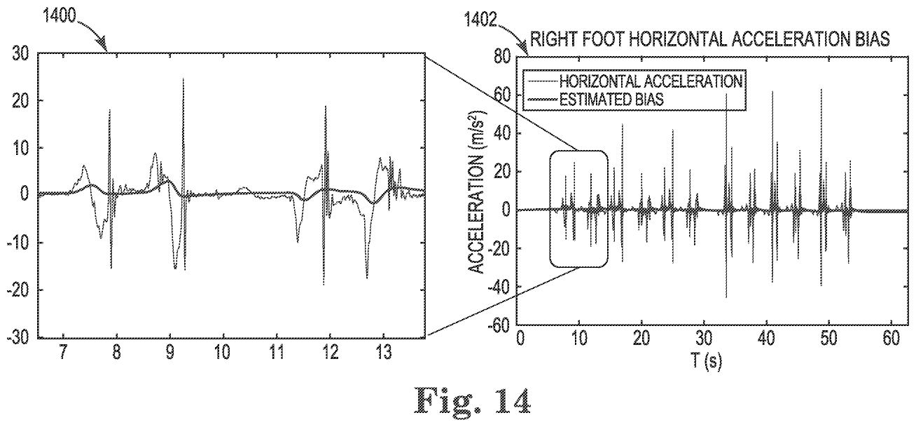

FIG. 14 is a diagram illustrating graphs 1400 and 1402 of the estimated bias from the nonlinear observer for the horizontal axis accelerations according to an example. This estimation shows that the bias changes as the foot accelerates and decelerates, which is due to the micro-electro-mechanical sensors having time-varying drift in the bias.

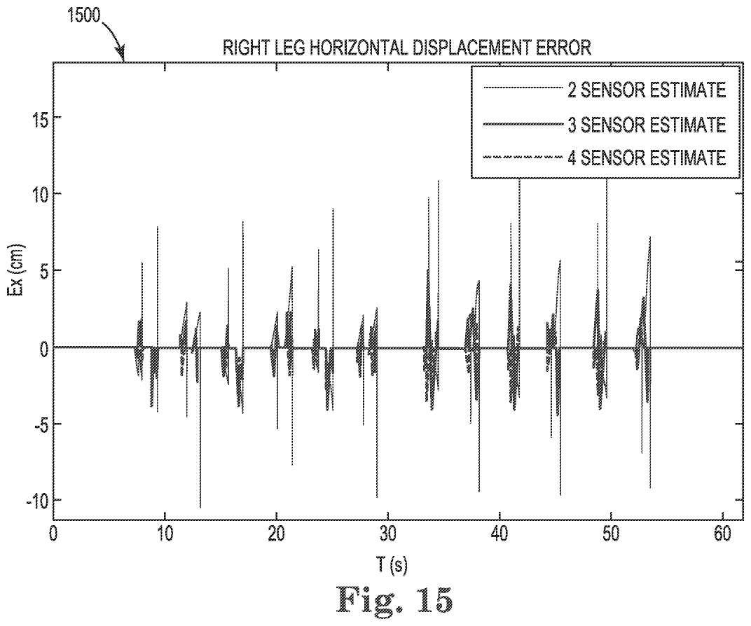

FIG. 15 is a diagram illustrating a graph 1500 of the horizontal displacement error for the right foot for the three observer-based methods (i.e., two-sensor integrator-based, four-sensor angle-based, and three-sensor polynomial angle-based) by comparing with the IR camera according to an example. As shown in this Figure, the angle mode has superior accuracy relative to the integration mode. The maximum error is 3.54 cm (7.63%) for the four-sensor mode and 5.12 cm (12.81%) for the three-sensor mode. On the other hand, the maximum error is 11.34 cm (28.35%) for the two-sensor integration mode. The maximum error becomes smaller if the stumbling part of the test is neglected. Maximum error is 2.94 cm (6.67%) for the four-sensor angle mode and 3.97 cm (8.94%) for the three-sensor angle mode, while the maximum error is 9.43 cm (19.52%) for the integration mode in just the first part of the test.

### F. Conclusions

This disclosure has presented three step length estimation methods using a nonlinear switched-gain observer based on measurements from IMUs 208 worn on leg segments. The observer estimates the orientation and measurement bias of each IMU 208. A switched-gain approach is used for the observer in which three piecewise working regions are utilized in each of which the output function is monotonic. The orientation of the IMU 208 and measurement bias are estimated in real-time, the bias and gravity components are removed from it to find the true linear accelerations, and then the accelerations are transformed to the intertial frame. Using this observer, three different approaches are presented for the step length estimation. In the first approach, it is assumed that IMUs 208 are used only on each shank, and the step lengths are found by integration of the linear inertial-frame accelerations of each leg from the start to the end of each step. In the second approach, it is assumed that there is one IMU 208 attached to each shank and a third IMU 208 is attached to one of the thighs. In the third approach, it is assumed that each leg has two IMUs 208, one IMU 208 on the shank and one IMU 208 on the thigh, and the step length may be estimated using the estimated angles of the limb segments and the geometry of the body posture. The second approach, which utilizes a sensor on only one of the two thighs, utilizes an approach to estimate the orientation angle of the other thigh by assuming symmetric motion between thighs during the swing phase.

The results of these estimators are compared to results from an IR camera motion capture system by performing tests of walking and stumbling in the forward and backward directions. The results show that the angles of the limbs are estimated very accurately, as verified by the IR camera. The integrator-based method is a simpler strategy and uses less resources due to the low number of IMUs 208. However, the angle-based method shows better accuracy in terms of step length estimation since it is not sensitive to the initial conditions and errors caused by double integration. The four-sensor method uses two sensors for the thighs and estimates both thigh angles from measurement of IMUs, while the three-sensor configuration estimates the thigh angle of one foot by fitting a polynomial to the estimated angle of the other foot. The three-sensor method shows comparable accuracy to the four-sensor mode while it does fall a little behind during more disturbances in stumbling experiments. Further, the accuracy of the three-sensor method may also fall when the gait of the subject is non-symmetric.

The nonlinear switched-gain observer disclosed herein is a helpful tool in the estimation of the orientation angles and bias values of IMU sensors, which may have problems in general with drifts and errors. Methods disclosed herein may be applied wherein IMUs are employed including navigation and stabilization applications. Two step length estimators are disclosed and may be used to help monitor activity and health of ordinary people, as well as athletes and patients.

Data has been collected from about 10-15 patients with PD or normal pressure hydrocephalus (NPH) who have gait and balance problems. The methods disclosed herein are quite accurate (>95%) in predicting the home activities as validated by video recordings, including predicting near falls. As a clinical decision support tool, once someone has been identified as a high fall risk, then a more detailed analysis of the sensor data could ensue to examine what specific factors put them at risk relative to, for example, similar age healthy controls or similar age PD patients who do not fall. If their steps are too small or asymmetric, they can be referred for PT gait therapy. If they react too slowly when a near fall occurs, they can undergo stochastic vestibular stim. If they are always tripping or falling at a certain time of day or location, they can undergo a medication review with their neurologist or have an occupational therapist review of their home.

The analysis of the recovery response from each subject can be used to identify specific kinematic deficits for targeted medical intervention. For example, deficits in step length could be improved with LSVT-BIG therapy while slow reaction times could be improved with stochastic vestibular stimulation. A patient may, for instance, have adequate step lengths in their recovery response, but their reaction time at home may be much slower than in clinic testing. Such a subject may benefit from an intervention to improve reaction time to an unexpected perturbation such as stochastic vestibular stimulation. It is also possible to more specifically impact aspects of the postural response with closed loop DBS (deep brain stimulation) paradigms wherein the recognition of the occurrence of specific risky activities can be used to automatically trigger specific neurostimulation to improve postural stability.

An example of the present disclosure is directed to a sensing and processing system, which includes a plurality of wearable sensing devices each including an inertial measurement unit (IMU) to be positioned on a subject and generate accelerometer signals and gyroscope signals. The system includes a processor to identify near-fall events, which are indicative of the subject nearly falling down, based on the generated accelerometer signals and gyroscope signals, and wherein the processor is to generate, for each of the near-fall events, subject response data indicative of a recovery response of the subject to recover from the near-fall event.

The plurality of sensing devices may include at least three sensing devices, each including an IMU, and wherein a first one of the sensing devices is configured to be positioned on a shank of a left leg of the subject, wherein a second one of the sensing devices is configured to be positioned on a shank of a right leg of the subject, and wherein a third one of the sensing devices is configured to be positioned on a chest of the subject. The plurality of sensing devices may include at least five sensing devices, each including an IMU, and wherein a fourth one of the sensing devices is configured to be positioned on a thigh of the left leg of the subject, and wherein a fifth one of the sensing devices is configured to be positioned on a thigh of the right leg of the subject.

The subject may be a person with at least one of Parkinson's Disease, hydrocephalus, and age-related postural instability. The processor may automatically extract, for each of the identified near-fall events, a data segment from the generated accelerometer signals and gyroscope signals corresponding to the near-fall event. The subject response data for each of the near-fall events may include a reaction time of the recovery response, and a number of steps and step lengths for the recovery response. The subject response data for each of the near-fall events may include chest velocity and acceleration data during the near-fall event.

The processor may use an activity classification decision tree to perform activity recognition and identify the near-fall events. The processor may use support vector machines to perform activity recognition and identify the near-fall events. The support vector machines may utilize chest acceleration or gyroscope data to identify bending, chest yaw rate data to identify turning, and leg acceleration or gyroscope data to identify whether the subject is taking steps. The support vector machines may utilize chest acceleration and gyroscope data to first identify an occurrence of bending, and then further classify a type of the bending as either a near-fall, fall, sit-to-stand transition, intentional bend, or a lie down event. Postural instability of the subject may be characterized by first identifying the occurrence of a near-fall event and then subsequently estimating a reaction time of the subject in taking a balancing step for recovery and counting a number and length of balancing steps taken. The processor may use a deep learning-based activity recognition method to identify the near-fall events. The processor may estimate tilt angles of a chest and leg segments of the subject based on the accelerometer signals and gyroscope signals, and estimate step lengths based on limb lengths of the subject and the estimated tilt angles. An occurrence of risky activities that pose a risk of falling including turning and sit-to-stand transitions may be used by the processor to provide feedback to a deep-brain-stimulation device so that real-time neuromodulation can be utilized to improve postural stability of the subject. Activity recognition and postural instability characterization may be used by the processor to tune neurostimulation parameters of a deep-brain-stimulation device implanted in the subject.

Another example of the present disclosure is directed to a method, which includes receiving, with a cloud-based processing system, inertial measurement unit (IMU) data from a plurality of IMU sensors, wherein the IMU data represents movement of a subject. The method includes identifying, with the cloud-based processing system based on the received IMU data, near-fall events indicative of the subject nearly falling down. The method includes generating, with the cloud-based processing system for each of the identified near-fall events, response data indicative of a recovery response of the subject to recover from the near-fall event.

In the method, the response data for each of the identified near-fall events may include a response time of the recovery response, and a number of steps and step lengths for the recovery response. The response data for each of the identified near-fall events may include chest velocity and acceleration data during the near-fall event.

Another example of the present disclosure is directed to a method, which includes positioning a plurality of sensing devices on a subject, wherein each of the sensing devices includes an inertial measurement unit (IMU). The method includes generating, with the IMUS, IMU data representing movement of the subject. The method includes wirelessly transmitting the IMU data from the sensing devices. The method includes processing the IMU data with a cloud-based processing system to identify near-fall events indicative of the subject nearly falling down, and extract data segments from the IMU data corresponding to the identified near-fall events.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. A sensing and processing system, comprising:
a plurality of wearable sensing devices each including an inertial measurement unit (IMU) to be positioned on a subject and generate accelerometer signals and gyroscope signals; and
a processor to identify near-fall events, which are indicative of the subject stumbling or nearly falling down, based on the generated accelerometer signals and gyroscope signals, and wherein the processor is to generate, for each of the near-fall events, subject response data indicative of a recovery response of the subject to recover from the near-fall event.

2. The sensing and processing system of claim 1, wherein the plurality of sensing devices includes at least three sensing devices, each including an IMU, and wherein a first one of the sensing devices is configured to be positioned on a shank of a left leg of the subject, wherein a second one of the sensing devices is configured to be positioned on a shank of a right leg of the subject, and wherein a third one of the sensing devices is configured to be positioned on a chest of the subject.

3. The sensing and processing system of claim 2, wherein the plurality of sensing devices includes at least five sensing devices, each including an IMU, and wherein a fourth one of the sensing devices is configured to be positioned on a thigh of the left leg of the subject, and wherein a fifth one of the sensing devices is configured to be positioned on a thigh of the right leg of the subject.

4. The sensing and processing system of claim 1, wherein the subject is a person with at least one of Parkinson's Disease, hydrocephalus, and age-related postural instability.

5. The sensing and processing system of claim 1, wherein the processor is to automatically extract, for each of the identified near-fall events, a data segment from the generated accelerometer signals and gyroscope signals corresponding to the near-fall event.

6. The sensing and processing system of claim 1, wherein the subject response data for each of the near-fall events includes a reaction time of the recovery response, and a number of steps and step lengths for the recovery response.

7. The sensing and processing system of claim 1, wherein the subject response data for each of the near-fall events includes chest velocity and acceleration data during the near-fall event.

8. The sensing and processing system of claim 1, wherein the processor is to use an activity classification decision tree to perform activity recognition and identify the near-fall events.

9. The sensing and processing system of claim 1, wherein the processor is to use support vector machines to perform activity recognition and identify the near-fall events.

10. The sensing and processing system of claim 9, wherein the support vector machines utilize chest acceleration or gyroscope data to identify bending, chest yaw rate data to identify turning, and leg acceleration or gyroscope data to identify whether the subject is taking steps.

11. The sensing and processing system of claim 9, wherein the support vector machines utilize chest acceleration and gyroscope data to first identify an occurrence of bending, and then further classify a type of the bending as either a near-fall, fall, sit-to-stand transition, intentional bend, or a lie down event.

12. The sensing and processing system of claim 1, wherein postural instability of the subject is characterized by first identifying the occurrence of a near-fall event and then subsequently estimating a reaction time of the subject in taking a balancing step for recovery and counting a number and length of balancing steps taken.

13. The sensing and processing system of claim 1, wherein the processor is to use a deep learning-based activity recognition method to identify the near-fall events.

14. The sensing and processing system of claim 1, wherein the processor is to estimate tilt angles of a chest and leg segments of the subject based on the accelerometer signals and gyroscope signals, and estimate step lengths based on limb lengths of the subject and the estimated tilt angles.

15. The sensing and processing system of claim 1, wherein an occurrence of risky activities that pose a risk of falling including turning and sit-to-stand transitions are used by the processor to provide feedback to a deep-brain-stimulation device so that real-time neuromodulation can be utilized to improve postural stability of the subject.

16. The sensing and processing system of claim 1, wherein activity recognition and postural instability characterization are used by the processor to tune neurostimulation parameters of a deep-brain-stimulation device implanted in the subject.

17. A method, comprising:
receiving, with a cloud-based processing system, inertial measurement unit (IMU) data from a plurality of IMU sensors, wherein the IMU data represents movement of a subject;
identifying, with the cloud-based processing system based on the received IMU data, near-fall events indicative of the subject nearly falling down; and
generating, with the cloud-based processing system for each of the identified near-fall events, response data indicative of a recovery response of the subject to recover from the near-fall event.

18. The method of claim 17, wherein the response data for each of the identified near-fall events includes a response time of the recovery response, and a number of steps and step lengths for the recovery response.

19. The method of claim 17, wherein the response data for each of the identified near-fall events includes chest velocity and acceleration data during the near-fall event.

20. A method, comprising:
positioning a plurality of sensing devices on a subject, wherein each of the sensing devices includes an inertial measurement unit (IMU);
generating, with the IMUs, IMU data representing movement of the subject;
wirelessly transmitting the IMU data from the sensing devices; and
processing the IMU data with a cloud-based processing system to identify near-fall events indicative of the subject nearly falling down, and extract data segments from the IMU data corresponding to the identified near-fall events.
